(19) United States
(12) Patent Application Publication
(10) Pub. No.: US 2010/0312051 A1
(43) Pub. Date: Dec. 9, 2010

(54) METHOD FOR TREATING STRESS URINARY INCONTINENCE AND SYMPTOMATIC PELVIC RELAXATION

(76) Inventor: Robert C. Brown, Fairhope, AL (US)

Correspondence Address: THE WEBB LAW FIRM, P.C., 700 KOPPERS BUILDING, 436 SEVENTH AVENUE, PITTSBURGH, PA, 15219, US

(21) Appl. No.: 12/867,311

(22) PCT Filed: Feb. 13, 2009
(86) PCT No.: PCT/US09/34039

§ 371 Date: Aug. 12, 2010

(30) Foreign Application Priority Data
Feb. 14, 2008 (US) 61/028648

(51) Int. Cl. A61F 2/04

(52) U.S. Cl. 600/37

(57) ABSTRACT

The invention relates to a method, anchors, mesh, tools, and kits for surgical procedures. The anchors are implanted in soft-tissue. Once implanted, a surgeon can manipulate an implantable supportive mesh, particularly the tension, through the same incision that the anchors were implanted. The anchors permit micro-adjustments to the tension. The tension on the supportive mesh can be secured through the same incision.

### SUMMARY OF THE INVENTION

The invention relates to a method, anchors, mesh, tools, and kits for performing surgical procedures. Examples of the surgical procedures include, but are not limited to, treating pelvic floor defects, stress urinary incontinence, female cystocele, female rectocele, female enterocele and other lifting applications.

The methods are minimally invasive procedures. For example, the procedures listed above are performed through a single small incision in the vagina. This is accomplished by the unique design of the anchors, mesh, tools and kits used in the inventive method. Anchors are implanted into connective tissue. The anchors have aperture(s) that allow micro-adjustment of the tension of the mesh by pulling filamentary elements of the mesh through the aperture(s).

The method comprises the steps of fixing at least two anchors to two different locations. Each anchor has a first aperture that allows free movement of a filamentary element of a mesh through the first aperture. Once the anchors are fixed, the tension of the mesh is adjusted by pulling the filamentary elements of the mesh through the aperture. Once a desired tension is reached, the tension is fixed. The tension is fixed by binding the filamentary elements, tapered portions or arms of the mesh, or by exposing a rough edge of the mesh to a portion of the soft tissue.

The anchors comprise a body having a head. The head is configured to facilitate insertion of the head into a tissue and retention of the head in the tissue once inserted. The body includes an aperture configured to allow free movement of a filamentary element through the aperture.

In another embodiment, the anchor comprises a distal end, a proximal end opposite the distal end, a non-tissue side, and a tissue side opposite the non-tissue side. A first aperture is positioned between the distal end and the proximal end. The first aperture extends through anchor from the non-tissue side to the tissue side. The aperture is configured to allow free movement of a filamentary element through the aperture.

Prior to implanting the anchor, an implantable mesh is drawn through the anchor's aperture. The mesh comprises a support section. A plurality of arms extends from the support section. Optionally, the mesh comprises a plurality of tapered portions that extend from the support section or the arms. A plurality of filamentary elements is connected to the support section, arms or tapered ends.

The mesh optionally may comprise a biodegradable sheath. The biodegradable sheath allows free movement through soft tissue and the anchor until the desired tension is obtained. The biodegradable sheath encases at least a portion of the support section, at least a portion of the arm and/or at least a portion of the filamentary element portion of the tapered portions of the mesh. The biodegradable sheath optionally may comprise a perforation. The perforation is configured to allow the removal of at least a portion of the sheath, preferably the portion of the sheath that encases a rough edge of the mesh, thereby exposing the rough edge of the mesh. The perforation may also be configured to allow removal of all or substantially all of the sheath from the mesh.

The anchors may be implanted using tools that are considered part of the invention. The tools comprise a body having a proximal end and a distal end. A handle is positioned at the proximal end of the body. Between the proximal and distal ends is a shaft, which is configured to allow insertion of an anchor into the desired location. In one embodiment, the shaft is substantially straight. In another embodiment, the longitudinal portion comprises a substantially straight portion and a curved portion. The angle between an imaginary line extending from a distal end of the curved portion and an imaginary line extending from the proximal end of the handle is between about 20 degrees to about 75 degrees.

Optionally, a blunt tip dissector is connected to the distal end of the body. The blunt tip dissector is configured to receive a base or proximal end of an anchor. The distal end of the body may also be configured to receive a base of the blunt tip dissector. In one embodiment, the base or proximal end of the anchor is configured to be mounted on the blunt tip dissector or the distal end of the body. The body optionally comprises a release trigger, which is operatively connected to the blunt tip dissector or the distal tip, wherein activation of the release trigger allows the connection between the blunt tip dissector and/or the anchor to be released.

The anchors may be implanted by use of inventive scissors. The scissors comprise a body having a cutting end and an operating end. A guide is connected to a portion of the body. The guide is configured to allow passage of the anchor and mesh through the guide while the scissors remain in the incision. Therefore, the operator would not need to remove the scissors from the incision. Instead, the anchor with mesh can be implanted via the guide by action of an advancing element. The anchor with mesh can be placed within the guide before the operator first uses the scissors. The implantation of the anchor can be controlled by any advancing element known to a person of ordinary skill in the art. An example of an advancing element is a rod-like structure positioned to exert force on the anchor through the guide when the rod-like structure is acted upon.

One embodiment of the invention is a kit comprising the mesh and a plurality of anchors. In this embodiment, the filamentary elements of the mesh are provisionally drawn through the anchors' apertures that correspond to each filamentary element. Finger grips are optionally positioned at terminal ends of the filamentary elements. The kits may further comprise an inserter and/or a tool, or a plurality of inserters or tools, each corresponding to a specific location for implanting an anchor.

### BRIEF DESCRIPTION OF THE DRAWINGS

FIG. 1 depicts a mesh implanted via the posterior repair procedure.

FIG. 2 depicts a mesh implanted via the anterior repair procedure.

FIG. 3 depicts anchors, mesh and tools for implanting mesh.

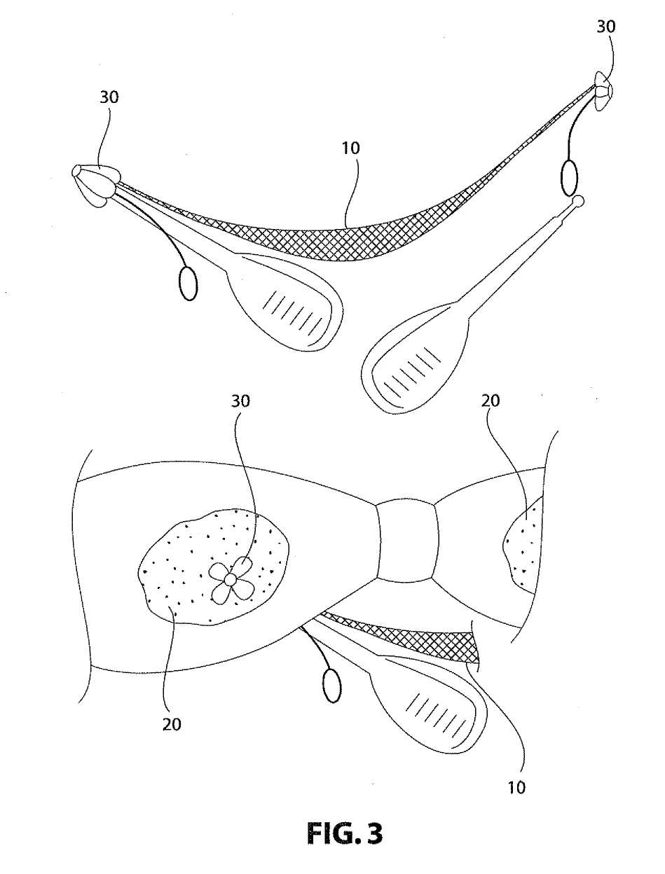

FIG. 4 depicts a pedal style anchor.

FIG. 5 depicts a pedal style anchor.

FIG. 6 depicts an arrowhead style anchor.

FIG. 7 depicts a geometric/flat style anchor with at least one aperture.

FIG. 8 depicts a geometric/flat style anchor with at least two apertures.

FIG. 9 depicts a geometric/flat style anchor together with an inserter and mesh.

FIG. 10 depicts a geometric/flat style anchor with a cone-shaped inlet and a cone-shaped outlet.

FIG. 11 depicts a flexible geometric/flat style anchor.

FIG. 12 depicts a hinged anchor.

FIG. 13 depicts a hook style anchor together with an inserter.

FIG. 14 depicts a mesh having four arms.

FIG. 15 depicts a mesh having six arms.

FIG. 16 depicts a mesh having two arms.

FIG. 17 depicts a mesh encased in a biodegradable sheath.

FIG. 18 depicts a biodegradable sheath with a perforation and a portion removed exposing a rough edge.

FIG. 19 depicts an embodiment of the scissors with guide, advancing element and anchor.

FIG. 20 depicts an embodiment of the scissors with guide, advancing element, anchor and a membrane.

FIG. 21 depicts an inserter.

FIG. 22 depicts a geometric/flat style anchor having an oval shape.

FIG. 23 depicts a tool with a cradle bearing a geometric/flat style anchor having an oval shape.

FIG. 24 depicts an insertion tool with an angled shaft and a hook style anchor.

FIG. 25 depicts a straight insertion tool and a curved insertion tool.

FIG. 26 depicts a geometric/flat style anchor having an oval shape and rough apertures.

### DETAILED DESCRIPTION OF THE INVENTION

The invention relates to methods, tools, mesh and anchors relating to using mesh in surgical applications. Generally, the anchors allow a surgeon to reduce the number of incisions made when implanting mesh or other supportive implant. In principle, this is accomplished by securing a supportive implant to a soft tissue with an anchor. The anchor is configured to allow it to be implanted into the soft tissue by passing partially or completely through the soft tissue, and permits the mesh, or a filamentary element of the mesh, to pass freely through an aperture of the anchor. The anchor is further configured to allow the mesh, or the filamentary element, to pass partially or completely through the soft tissue and return to the operator side. This allows a surgeon to perform micro-adjustments to the tension of the mesh without having to create a second incision to access the mesh on the opposite side of the soft tissue.

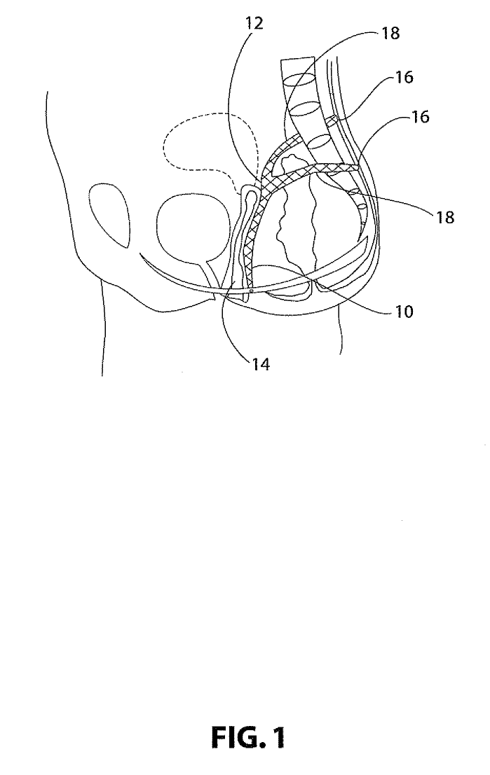

One embodiment of the invention is a minimally invasive surgical procedure for posterior repair of vaginal prolapse. The procedure is performed through a single incision in the vaginal wall. A midline vaginal incision, approximately 4 cm in length, is made in the posterior vagina, over the rectum. Fluid is injected into the recto-vaginal space to temporarily separate the vagina from the rectum. Mesh 10 is inserted into the recto-vaginal space through the incision and positioned appropriately to provide the desired support. The mesh is attached to the apex 12 of the vagina, and the vaginal opening 14 at the perineal body. The method of attaching the mesh can include suturing the mesh to the attachment area or pushing the rough edge of the mesh into the attachment area. Anchors are bilaterally implanted into the sacrospinus ligament 16. Thereafter, the tension of the mesh 10 is adjusted by pulling the mesh's filamentary elements 24 through the anchors' apertures. Once the desired tension is reached, the desired tension is fixed by binding opposite ends of filamentary elements 24, tapered ends 106 or arms 104 together, or by pushing or exposing the rough edge 126 of the mesh 10 into soft tissue. The desired tension relates to the support provided to the vagina. As the filamentary elements 24 are drawn through the anchors' apertures, the vagina is pulled up into a raised position. Once an operator determines that the vagina's position is appropriate, the tension of the mesh 10 should be fixed. The implanted mesh 10 is illustrated in FIG. 1.

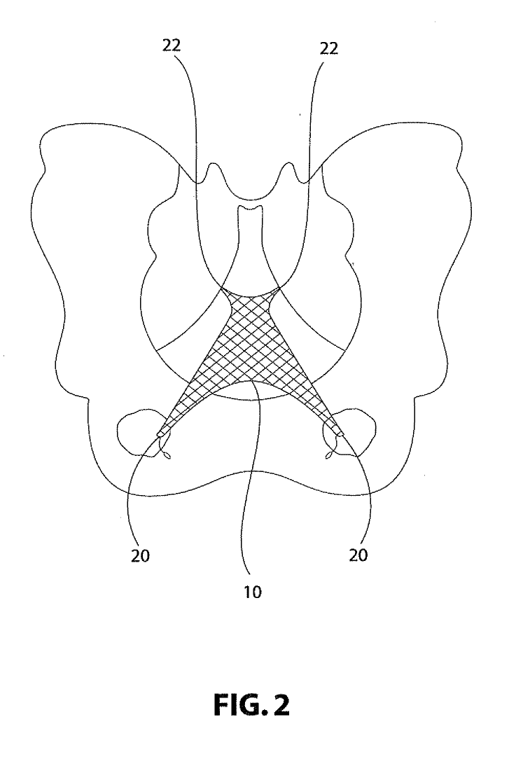

FIG. 2 depicts a minimally invasive procedure for the anterior repair of vaginal prolapse. This embodiment is analogous to the posterior repair of vaginal prolapse because it also suspends a prolapsed vagina. However, in this procedure, the mesh 10 is placed on the anterior wall of the vagina, and the anchors are implanted in the obturator membrane. This method is performed through an incision in the anterior wall of the vagina at the urethrovesical junction. The incision is approximately 4 cm in length. The mesh 10 is placed along the anterior side of the vagina, and is optionally attached to the apex of the vagina, and the vaginal opening at the perineal body. The method of attaching the mesh can include suturing the mesh to the attachment area or pushing the rough edge of the mesh into the attachment area. Two anchors are bilaterally implanted into the obturator membranes 20, and two other anchors are bilaterally implanted in the arcus tendinius fascia pelvis 22. Thereafter, the tension of the mesh 10 is adjusted by pulling filamentary elements 24 through the anchors' apertures. Once the desired tension is reached, the desired tension is fixed by binding the filamentary elements 24, tapered ends 106 or arms 104 together, or by pushing the rough edge 126 of the mesh 10 into soft tissue. The desired tension relates to the support provided to the vagina. As the filamentary elements 24, are drawn through the anchors' apertures, the vagina is pulled up into a raised position. Once an operator determines that the vagina's position is appropriate, the tension of the mesh 10 should be fixed.

Another embodiment of the invention combines the anterior and posterior repair procedures. In this embodiment, the vagina is supported anteriorally and posteriorally. Anchors are implanted bilaterally in the sacrospinus ligaments 16, obturator membranes 20, and arcus tendinius fascia pelvis 22.

Another embodiment of the invention is a repair for incontinence. The mesh 10 is inserted via a vaginal incision and positioned under the mid-urethra in this procedure. Anchors are bilaterally implanted into and within the obturator membranes 20. The mesh 10 is adjusted to a desired tension by pulling the filamentary elements 24 through apertures within the anchors. Once the desired tension is reached, the desired tension is fixed by binding the filamentary elements 24, tapered ends 106 or arms 104 together, or by exposing or contacting the rough edge 126 of the mesh 10 with a portion of the soft tissue.

These procedures are only exemplary procedures for using the anchors, mesh, tools and scissors discussed below. A person of ordinary skill in the art would readily identify different uses for these anchors, mesh, tools and scissors. Other uses include treating female cystocele, female rectocele, female enterocele, and performing other lifting or supporting applications. Therefore, the application of the anchors, mesh, tools and scissors are not limited to the methods discussed above.

The anchors developed to perform these types of procedures comprise a body. The body includes a head, which is configured to facilitate insertion of the head into a tissue and retention of the head in the tissue once inserted. The head can be configured so that it is retained by pushing the head completely through the tissue, or by pushing it partially through the tissue thereby leaving the head embedded within the tissue. The anchors comprise a channel or an aperture, which is positioned within the body of the anchor. The channel or aperture is configured to allow free movement of an arm, filamentary element or tapered end of a mesh through the channel. In one embodiment, the anchor comprises two channels, an inlet and an outlet.

There are various configurations of anchors that can be used for incontinence repair, anterior repair of vaginal prolapse or posterior repair of vaginal prolapse. These anchors can also be used in other surgical procedures involving securing artificial or biological material to a soft tissue. Some examples of suitable anchors are shown in the figures and described below.

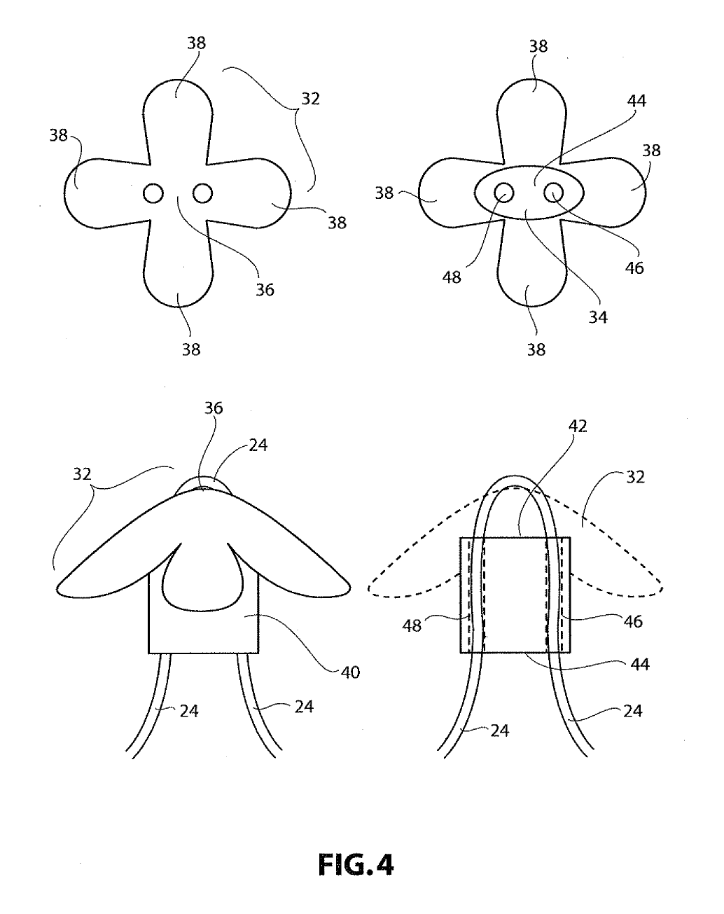

FIG. 4 depicts a pedal style anchor. In this embodiment, the anchor comprises a body having a head 32. The head 32 comprises a center 36 and a plurality of pedals 38 extending from a center 36 of the head 32. In one aspect of this embodiment, the anchor comprises at least four, at least five, or at least six pedals 38. The anchor further comprises a base 40 extending from the head 32. The base 40 has a bottom 44 and a top 42, wherein the top 42 is positioned near the head 32. The base 40 has an inlet channel 46 and an outlet channel 48. The inlet and outlet provide a passage for a filamentary element 24 of the mesh 10 from the inlet 46, through the base 40, and out the outlet 48. Optionally, the pedals 39 are constructed of a resiliently flexible material, so that once implanted in a membrane, the pedals move from a retracted position to an extended position by virtue of the tension from the mesh 10.

In one aspect of this embodiment, the base comprises one channel. In this aspect, the channel has a curved shape, an inlet positioned on the bottom of the base 44, and an outlet positioned on the bottom of the base 44.

In another aspect of this embodiment, as depicted in FIG. 4, the base comprises two channels 46 and 48. In this aspect, the inlet 46 can have a substantially straight shape, and it provides communication between the bottom 44 and the top 42. The inlet 46 passes through from the bottom 44, through the base 40 to the top 42. The outlet 48 can also have a substantially straight shape, and it provides communication between the bottom 44 and the top 42. The outlet 48 passes through from the top 42, through the base 40 to the bottom 44. Optionally, the head 32 may comprise an opening at the center 36 thereby exposing the top 42 of the base 40, and optionally the filamentary element 24 of the mesh 10.

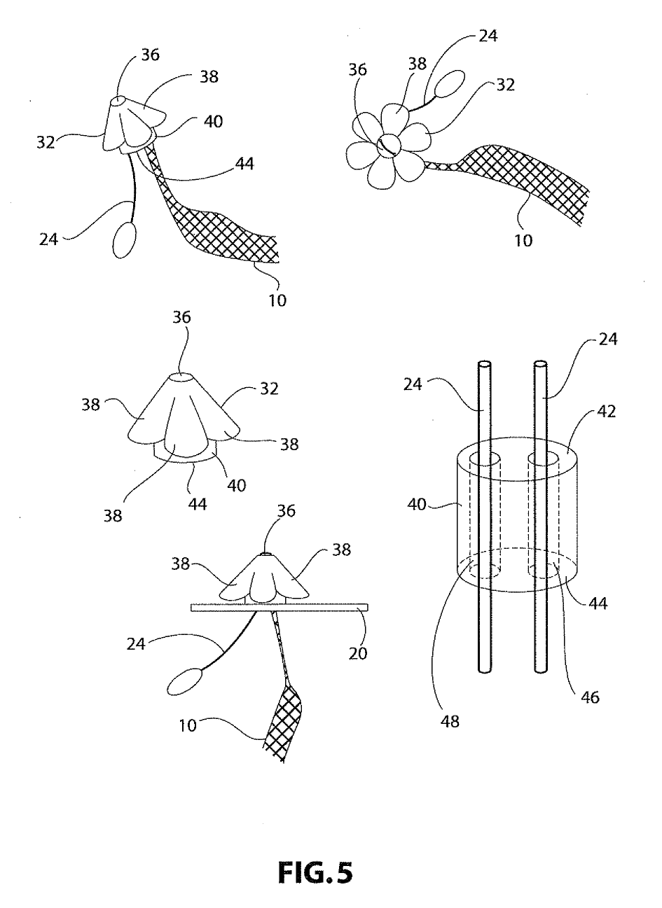

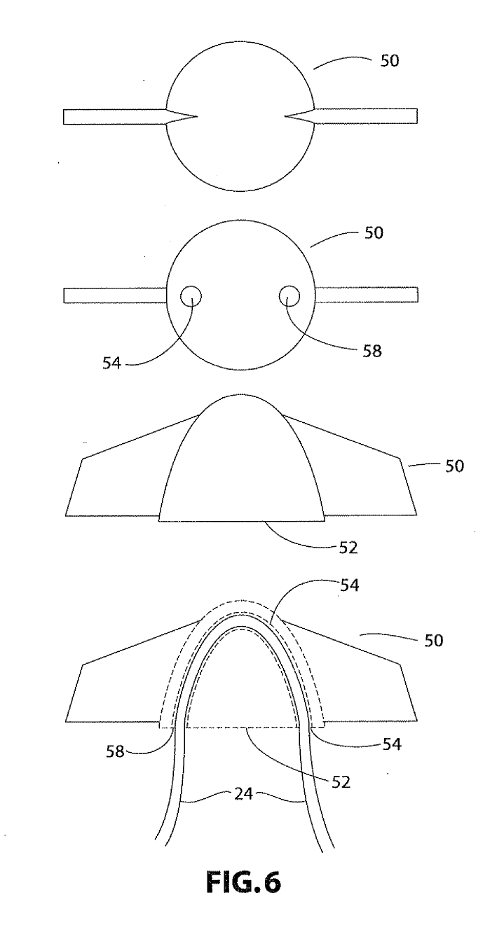

FIG. 6 illustrates another embodiment of an anchor. This embodiment generally comprises an arrowhead shape. The anchor comprises a head 50 in an arrow head shape, or a T-shape. The anchor further comprises a proximal end 52 and at least one channel 54 having an inlet 56 and an outlet 58. In one aspect of this embodiment, the channel 54 has a curved shape so that the inlet 56 and an outlet 58 are positioned in the proximal end 52 of the anchor. In another aspect of this embodiment, the body comprises two channels similar to the ones depicted in FIG. 5 and described above. In one aspect of this embodiment, the anchor may be adapted to receive a blunt tip dissector at a distal tip of the head 50.

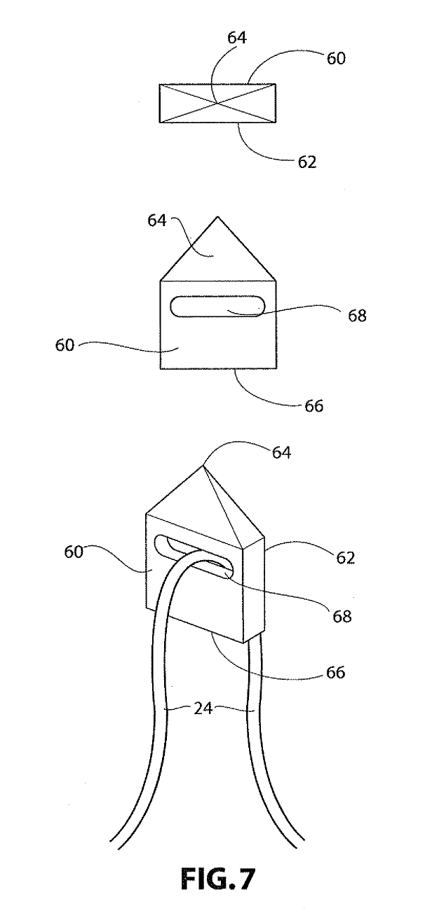

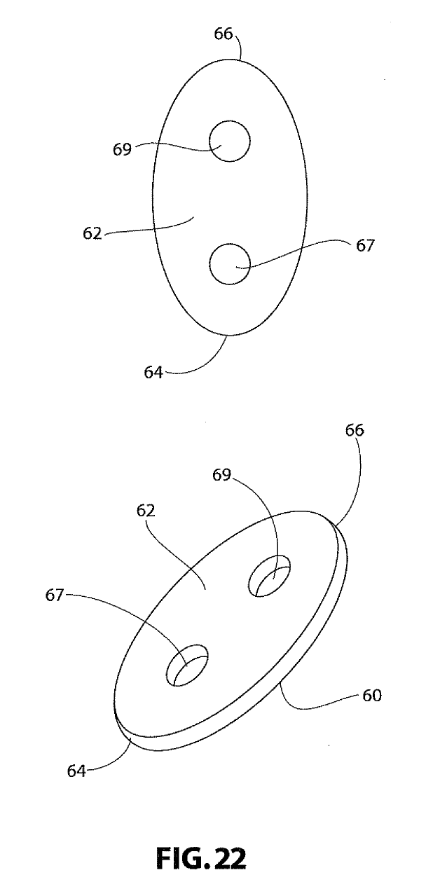

FIGS. 7-12 and 22 depict examples of geometric/flat style anchors. In these embodiments, the anchor comprises a tissue side 60, a non-tissue side 62 opposite the tissue side 60, a distal end 64 at one end of the anchor, a proximal end 66 opposite the distal end 64, and at least one aperture 68 positioned between the distal end 64 and the proximal end 66. The distal end 64 may be pointed or adapted for cutting soft tissue, or may be curved. The proximal end 66 may be substantially straight or may be curved. If both the proximal and distal ends are curved, the anchor may generally have an oval shape or substantially oval shape, such as the anchor depicted in FIG. 22. The aperture 68 provides communication between the tissue side 60 and the non-tissue side 62. The aperture 68 is configured to allow free movement of a filamentary element 24. The tissue side 60 may be substantially flat. The non-tissue side 62 may be substantially flat. The distal end 64 may be configured to advance the anchor through a tissue.

Examples of tissue include soft tissue, such as ligaments, tendons and other connective tissue. An operator inserts this anchor by advancing the insertion end through the tissue. Once the entire anchor has been advanced through the entire thickness of the tissue, the anchor is laid flat against the tissue thereby resisting removal of the anchor from the tissue. One aspect of this embodiment is an anchor designed so that the length between the cutting end and the proximal end is greater than the width between the two sides. Another aspect of this embodiment is a configuration that would resist removal when the anchor is turned approximately 90 degrees at the distal side of the tissue. During implantation, the filamentary element 24 is advanced with the anchor to the distal side of the tissue. Upon implantation, the filamentary element 24 is positioned so it passes through the incision in the tissue, through the tissue toward the distal side of the tissue, over a portion of the anchor, proximally through the aperture 68, and proximally through the tissue. Therefore, the finger grips 108 or terminal ends of the filamentary element 24 are positioned at the proximal side or operator side of the tissue.

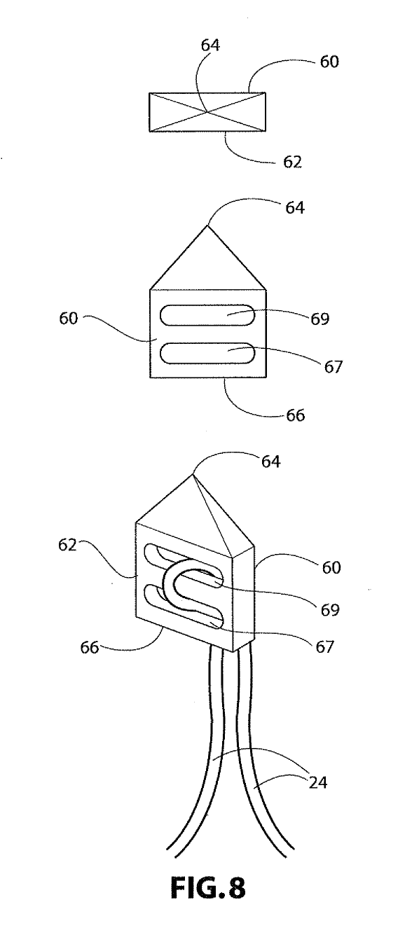

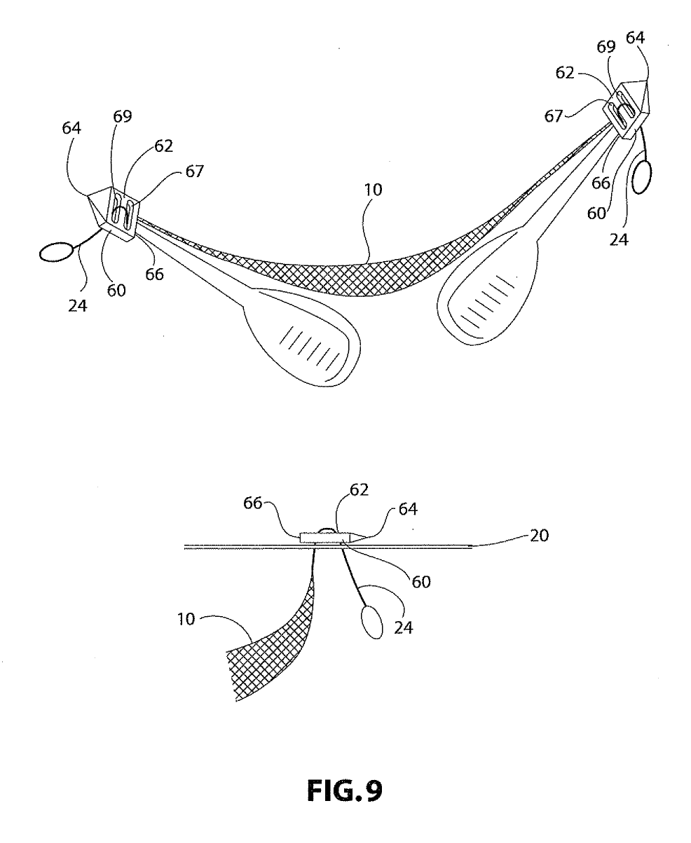

FIG. 8 depicts another embodiment of a geometric/flat anchor. This embodiment comprises two apertures, an inlet 67 and an outlet 69. Both the inlet 67 and the outlet 69 provide communication between the tissue side 60 and the non-tissue side 62. A filamentary element 24 can be passed through the inlet 67 from the tissue side 60. The filamentary element 24 passes over a portion of the non-tissue side 62 of the anchor, preferably in contact with that portion and preferably the portion of the anchor between the two apertures. The filamentary element 24 continues to extend through the outlet 69 by entering the outlet 69 at the non-tissue side 62, extending through the anchor and exiting the outlet 69 at the tissue side 60. This particular configuration provides an additional advantage when flattening the anchor after the anchor has been passed through the membrane 20. Once the anchor is passed through the membrane 20, by pulling on the filamentary element 24, the distal end 64 of the geometric/flat anchor will be pulled towards the membrane 20 so that the tissue-side 60 of the anchor will lay substantially flat against the membrane 20, as illustrated in FIG. 9. This action is created because, by pulling the portion of the filamentary element 24 that extends from the outlet 69, the distribution of force on the anchor causes the distal end 64 to move towards the membrane 20.

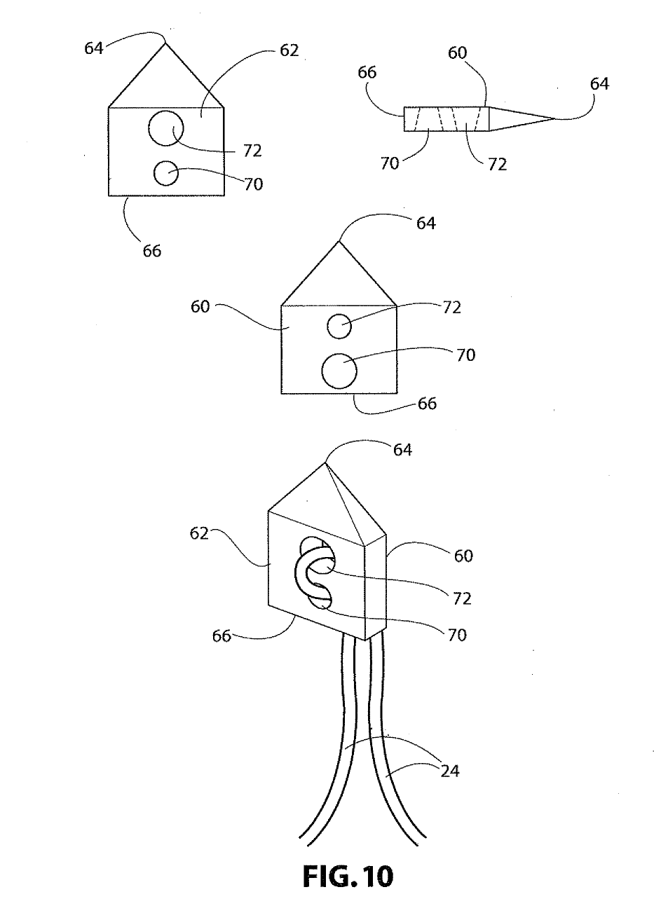

FIG. 10 depicts another embodiment of the geometric/flat anchor. In this embodiment, the anchor further comprises a cone-shaped inlet 70 and a cone-shaped outlet 72. The cone-shaped inlet 70 comprises a tissue side 60 diameter that is larger than the non-tissue side 62 diameter of the cone-shaped inlet 70. The cone-shaped outlet 72 comprises a non-tissue side 62 diameter that is larger than the tissue side 60 diameter of the cone-shaped outlet 72. The cone-shaped inlet 70 and the cone-shaped outlet 72 can replace any inlet or outlet, or any aperture in any of the geometric/flat anchors.

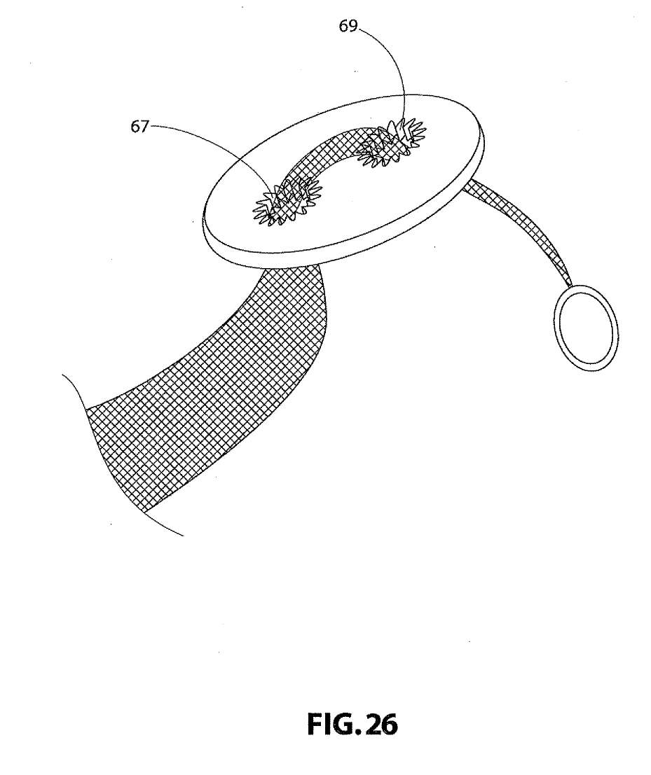

In another embodiment, as depicted in FIG. 26 the apertures 67 and 69 comprise a rough edge. The rough edge may be barbed. In this embodiment, the anchor's rough edges would catch a mesh 10 or a rough edge 126 of a mesh 10, thereby securing the tension of the mesh 10. The rough edge can be applied in any embodiment comprising a channel or an aperture including in conjunction with the cone-shaped inlet 70 and cone-shaped outlet 72. Preferably, the mesh 10 used with this embodiment comprises a biodegradable sheath 120 and a perforation 122, such as the ones discussed below.

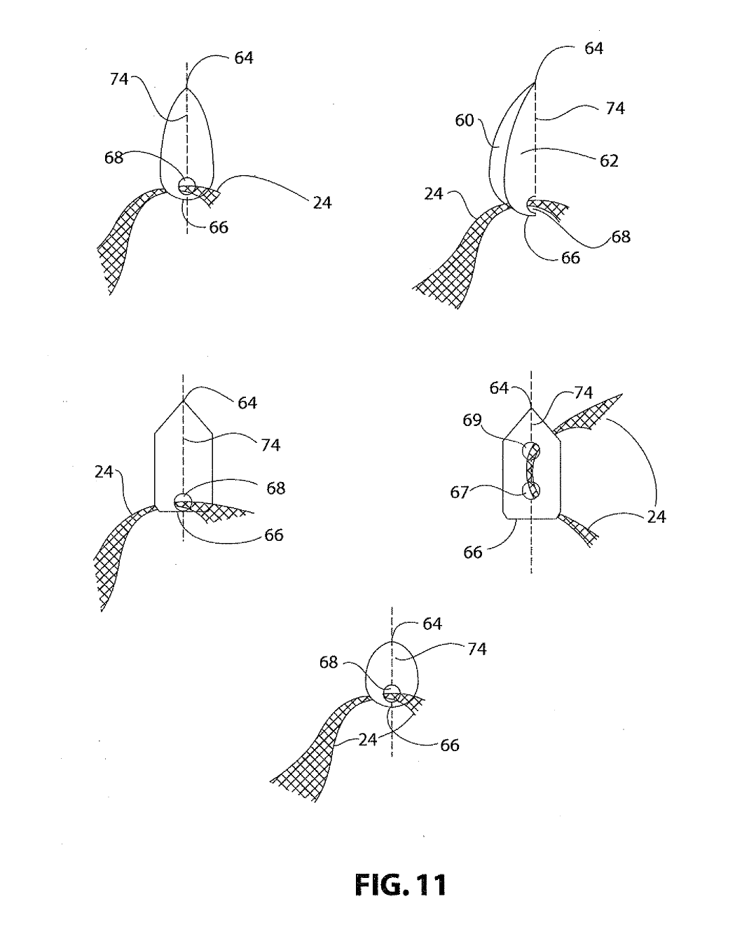

FIG. 11 depicts another embodiment of the geometric/flat anchor. In this embodiment, the anchor is constructed of a resilient flexible material. Suitable materials for flexible surgical anchors are known to those skilled in the art. This embodiment further comprises an imaginary flex-line 74. Prior to implantation, the anchors can be folded along the imaginary flex-line 74, thereby forming a temporarily folded anchor. The temporarily folded anchor can be pushed through the membrane 20. Once the anchor passes through the entire membrane 20, the anchor unfolds by virtue of its resilient nature. The operator, by pulling on the filamentary element 24, can cause the anchor to lay substantially flat against the membrane.

In another embodiment, an example of which is depicted in FIG. 22, the geometric/flat style anchor comprises a general oval shape with at least one aperture, or two apertures 67 and 69. By virtue of its shape, this geometric/flat style anchor does not present any sharp edges.

In the embodiment of the geometric flat anchor comprising one aperture, the filamentary element 24 is passed over a portion of the non-tissue side 62 of the anchor, through the aperture 68 towards the tissue side 60 of the anchor. The filamentary element 24 comprises a mesh 10 at a first terminal end of the filamentary element 24. The first terminal end of the filamentary element 24 is associated with the non-tissue side 62 of the anchor. Optionally, the filamentary element 24 further comprises a finger grip 108 positioned on a second terminal end of the filamentary element 24, wherein the second terminal end is opposite the first terminal end. The second terminal end is associated with the tissue side 60. The mesh may further comprise a biodegradable sheath 120 that encases at least a portion of the mesh 10. The biodegradable sheath 120 may comprise a perforation 122 configured to exposure a rough edge 126 of the mesh 10, which, as described below, can be used to secure the tension of the mesh.

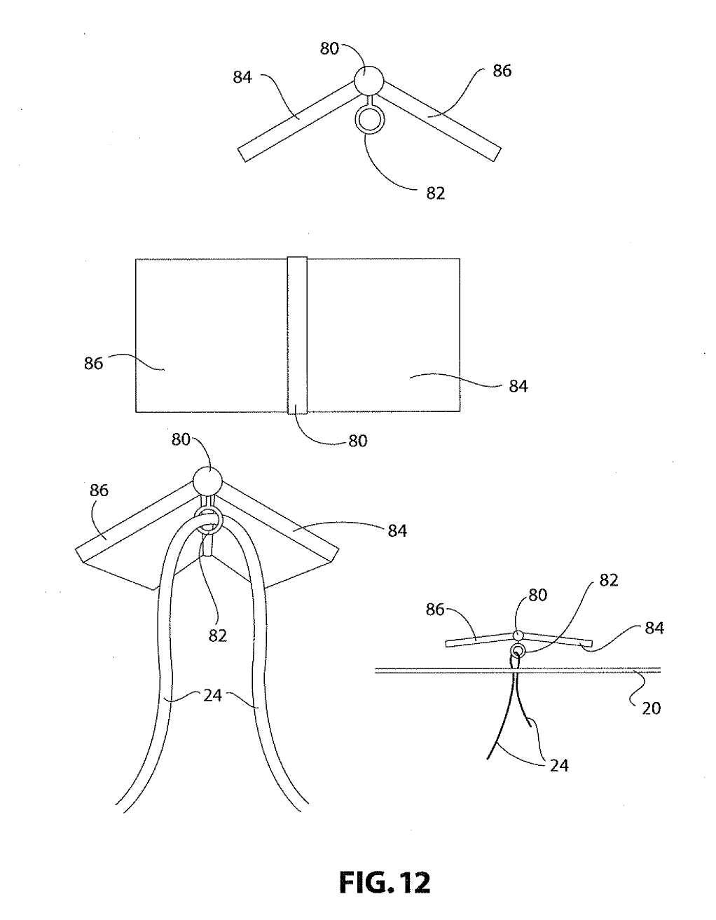

FIG. 12 depicts an example of a hinged anchor. In this embodiment, the anchor comprises a hinge 80 that connects at least two wings 84 and 86. The anchor further comprises an aperture 82 configured to allow free movement of a filamentary element 24 through the aperture 82. The hinge 80 of the anchor may be configured to cut soft tissue. As the anchor is being advanced through the tissue, the wings 84 and 86 are in a retracted position. Once through the tissue, the wings 84 and 86 extend to an extended position when the operator acts upon the filamentary element 24. In the extended position, the wings 84 and 86 provide resistance so that the anchor remains implanted in the membrane 20. The movement of the wings 84 and 86 occurs at the hinge 80. Prior to implantation, the filamentary element 24 is extended through the aperture 82. The hinged anchor with mesh attached is pushed to a distal side of the membrane 20. Consequently, a portion of the filamentary element 24 and/or mesh 10 extend though at least a portion of the membrane 20, through the aperture 82, and back through the tissue to the proximal side or operator side of the membrane 20. Therefore, an operator can manipulate the filamentary element 24 that extends from the aperture 82 on the proximal side of the membrane 20.

In embodiments of the geometric flat anchor comprising two apertures, the filamentary element passes through the inlet 67 over at least a portion of the non-tissue side 62 of the anchor and through the outlet 69. Preferably the filamentary element 24 passes over a portion of the anchor positioned between the inlet 67 and outlet 69. The filamentary element 24 comprises a mesh 10 positioned at a first terminal end of the filamentary element 24. The mesh 10 is associated with the tissue side 60 of the anchor. The filamentary element 24 may optionally also comprise a finger grip 108 positioned at a second terminal end. The finger grip 108 is associated with the tissue side 60.

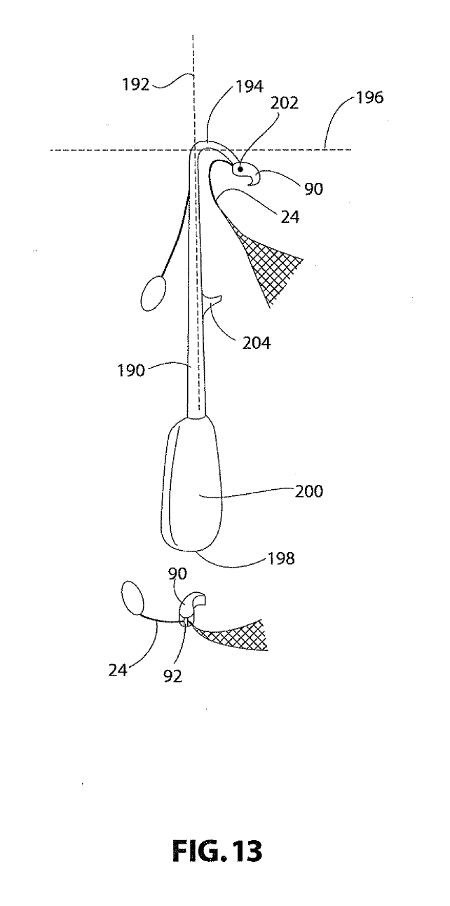

FIG. 13 depicts a hook styled anchor. In this embodiment, the anchor comprises a curved tip 90 and a channel 92. The curved tip 90 is configured to penetrate soft tissue when manual pressure is applied. The channel 92 is similar to the channels discussed above for the other embodiments of the anchor.

The anchors are constructed of biologically inert materials. Examples of such materials include plastics, stainless steel, titanium, or other non-reactive materials that can co-exist within a tissue. Optionally, the anchors are constructed of biodegradable materials.

The anchors may be formed by any means known to a person of ordinary skill. A preferred method of forming the anchors is by injection molding, wherein each anchor would be formed in a single mold. Therefore, each part of the anchors would be integrally formed.

The anchors are configured to allow an operator to adjust the tension of an implantable supportive mesh 10. The mesh 10 can be constructed of any biologically compatible synthetic material, or any natural material such as autologoues, allografts, xenografts, tissue engineered matrixes, or combinations thereof. An exemplary synthetic material is polypropylene mesh manufactured by Ethicon, Inc., a Johnson & Johnson company located at Somerville, NJ, United States of America. The mesh 10 may also be constructed of combinations of synthetic and natural materials.

Generally, the mesh 10 comprises a support section 100 and a filamentary element 24 extending from the support section. The mesh 10 may optionally comprise an arm 104 positioned between the filamentary element 24 and support sections. The arm 104 is an extension of mesh 10 that is thinner than the support section. Generally, the arm 104 is made of the same material as the mesh 10. Towards its terminal end, the arm 104 optionally may comprise a tapered portion 106. The filamentary elements 24 may optionally comprise a finger grip 108 positioned at a terminal end of the filamentary element 24.

The support section 100 is configured to support a particular tissue, such as a vagina.

In one embodiment, the support section 100 comprises a plurality of filamentary elements 24 extending from it. In this embodiment, each filamentary element 24 optionally may have a finger grip 108 at or near its terminal end. The filamentary element 24 may be a suture, or a rolled or folded portion of mesh.

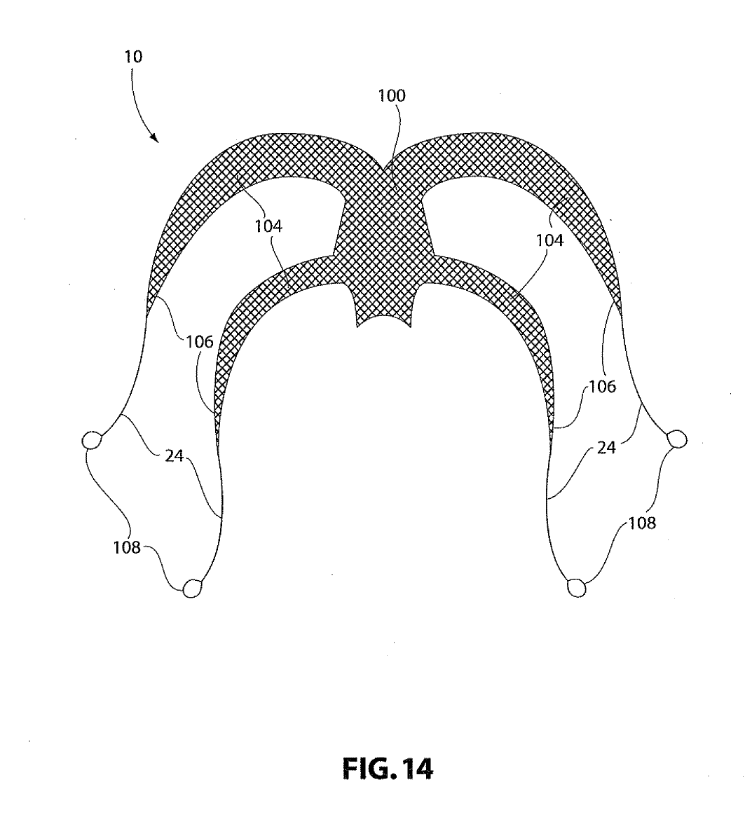

One embodiment is a mesh configured for the anterior repair procedure for vaginal prolapse. In this embodiment, the mesh comprises a support section 100, optionally up to four arms 104, four filamentary elements 24 extending from the support in section 100 at a corresponding arm 104, optionally up to four tapered portions 106 corresponding to each arm 104, and optionally up to four finger grips 108 corresponding to each filamentary element 24. The mesh generally has an “X” shape. The support section 100 may have a general square or rectangular shape, and the extensions extend from or near the corners of the support section. An example of this embodiment is illustrated in FIG. 14.

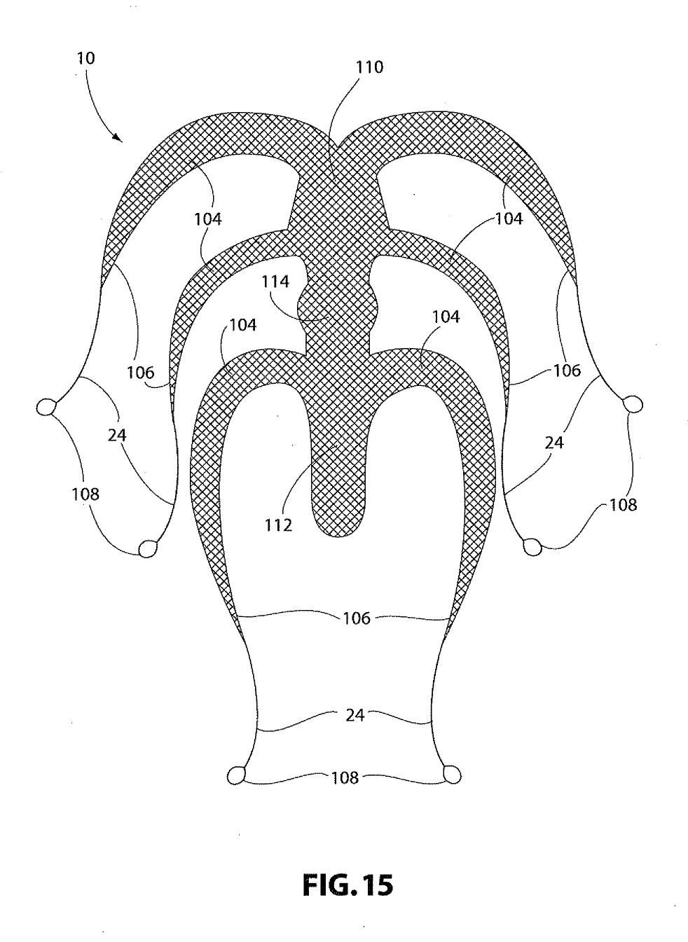

Another embodiment is a mesh configured for the total repair procedure. In this embodiment, the mesh comprises a support section 100, optionally up to six arms 104 extending from the support section 100, six filamentary elements 24 extending from the support section 100 at a corresponding arm 104, optional up to six tapered portions 106 corresponding up to each arm 104, and optionally up to six finger grips 108 corresponding to each filamentary element 24. Three of the filamentary elements 24 are paired with the other three and extend from opposite locations on the support section 100. In one aspect of this embodiment, the mesh comprises a first support section 110 and a second support section 112. The first 110 and second 112 support sections are connected by a connector portion 114. Four filamentary elements 24 extend from the first support section 110, and two filamentary elements 24 extend of the second support section 112. An example of this embodiment is depicted in FIG. 15.

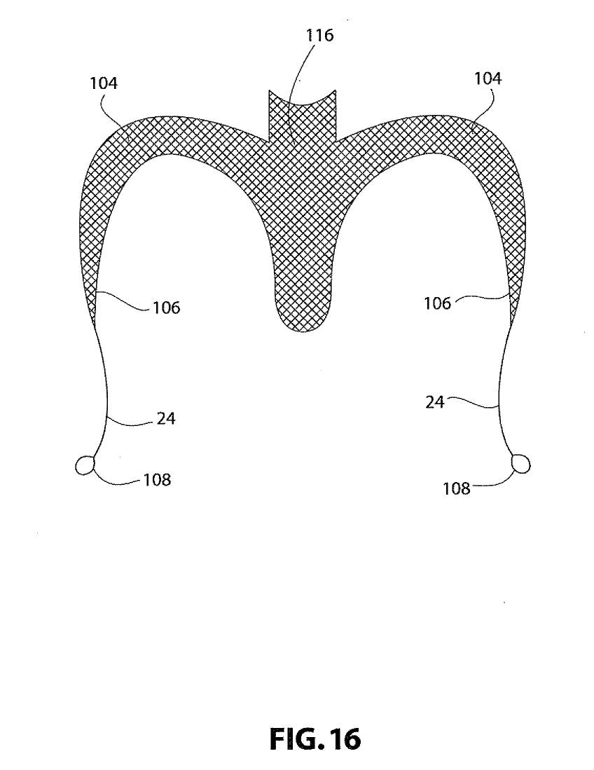

Another embodiment is a mesh for the posterior repair procedure for vaginal prolapse. In this embodiment, the mesh comprises a support section 100 and two filamentary elements 24. The mesh and arm can form a “Y” shape. An example of this embodiment is depicted in FIG. 16.

The mesh 10 may further comprise a biodegradable sheath 120. The sheath 120 encases at least a portion of the mesh 10, at least a portion of the arms 104, at least a portion of the tapered portion 106, and/or at least a portion of the filamentary element 24. Suitable materials for the sheath include, but are not limited to, PDS, Vicryl™ or Monocryl™. A mesh comprising a sheath 120 can be constructed in any manner known to a person of ordinary skill in the art. For example, a mesh encased in a sheath 120 can be constructed by creating the sheath 120 and pulling the mesh through it, folding or tapering the mesh inside the sheath 120.

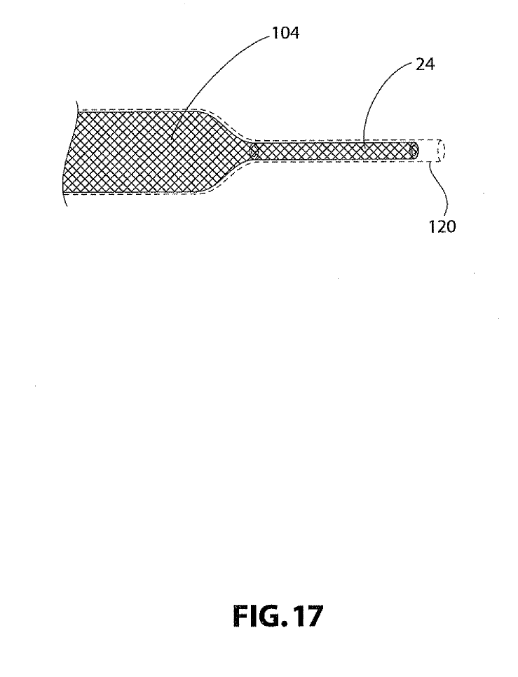

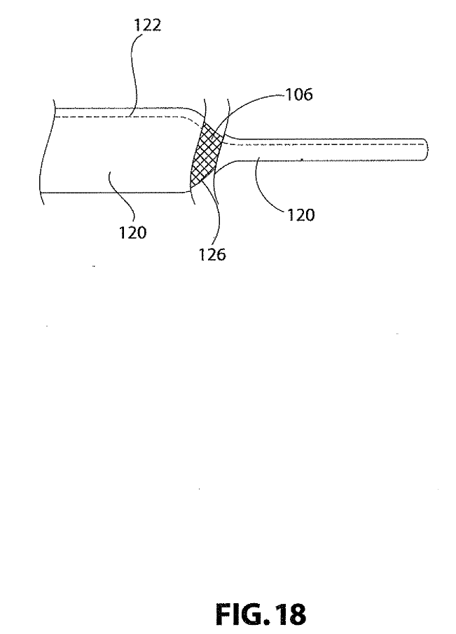

The biodegradable sheath 120 optionally may comprise a perforation 122. In this embodiment, the perforation is configured to expose at least a portion of the mesh 10, support section 100, arms 104, tapered portions 106, and/or filamentary elements 24 when a perforated section 122 is removed. The perforation 122 may be configured to expose a portion of a rough edge 126 of the mesh 10, support section 100, filamentary element 24, arm 104 and/or tapered portion 106 when a perforated section 122 is removed. It may be configured to expose the entire mesh 10, support section 100, filamentary element 24, arm 104 and/or tapered portions 106 encased by the sheath 120 when a perforated section 122 is removed. An example of one aspect of this embodiment is illustrated in FIGS. 17 and 18.

In another embodiment, the mesh further comprises an anchor, such as an anchor described above. The anchor is positioned on a filamentary element 24, wherein the filamentary element 24 passes through the aperture or apertures of the anchor.

Once the anchors are implanted, the mesh is adjusted to a desired tension. The adjustment is made via manual manipulation of the filamentary elements 24. The operator pulls the filamentary elements 24 through the aperture until the desired tension is reached, and then fixes the desired tension.

The desired tension can be fixed by various methods or device. For example, the extensions can be bound together by suturing, stapling, tying, clamping or cinching elements or ends together.

In one embodiment, the tension is fixed by pulling the extensions through a clamping device. The clamping device comprises a channel and a moveable clamp positioned within the channel. The moveable clamp has an open position and a closed position. The moveable clamp is operatively connected to a spring to bias the movable clamp to the closed position. During operation of a button on the clamping device, the spring is compressed placing the moveable clamp in the open position. When the moveable clamp is in the open position, the extensions can be passed through the channel until the desired tension is reached. Once the desired tension is reached, the button can be released, thereby moving the moveable clamp into the closed position and causing the clamp, by action of the spring, to apply pressure on the extensions that are positioned within the channel in proximity of the moveable clamp. In the closed position, the extensions are secure and cannot be moved; therefore, the tension is fixed.

The tension of the mesh 10 can also be fixed by exposing or contacting the rough edges 126 of the mesh 10 with tissue. The rough edges 126 of the mesh 10 have the ability to embed into soft tissue and fix the tension of the mesh. One specific example of fixing the tension is, just before or once the desired tension is reached, the perforated section 122 on the sheath 120 is removed, thereby exposing at least one rough edge 126. The exposed rough edge 126 is then pushed into the surrounding soft tissue or placed in contact with surrounding soft tissue. Further adjustments to the tension can be made after the perforated section 122 on the sheath 120 has been removed. Alternatively, the aperture of the anchor can have a rough surface, such as a barbed surface. In this embodiment, exposing a mesh to the rough surface in the aperture would secure the tension. In another embodiment where the aperture is cone-shaped inlet 70 or cone-shaped outlet 72 and has a rough surface 67 or 69, the filamentary element 24 pass through the aperture more easily in one direction, preferable a direction that tightens the tension. In this embodiment, it is preferred that the filamentary element 24 be encased in a sheath 120 thereby allowing easier manipulation of the tension until the sheath 120 covering the filamentary element 24 is removed via the perforation 122.

The anchors can be implanted using tools adapted to receive the base or proximal ends of the anchors. Generally, the tool comprises a shaft having a proximal end, a handle attached to the proximal end of the shaft, and a tip at the distal end of the shaft. The shaft may be substantially straight or may include one or more curved sections.

In one particular aspect, as shown in FIG. 13, a substantially straight section 190 is connected to the handle 200. The substantially straight section 190 has a longitudinal axis 192. A curved section 194 is connected to the substantially straight section. The curved section 194 curves away from the longitudinal axis 192 towards an axis perpendicular 196 to the longitudinal axis 192. From this point, the curved section 194 curves towards the longitudinal axis 192 and in the general direction of a proximal end 198 of the tool. The curved section 194 terminates at a tip 202. The curved section 194 only curves in a substantially two dimensional plane. In one embodiment, the hook style anchor 90 is preferably capable of removable attachment to the tip 202 of the curved section 194. This embodiment is particularly useful in implanting the anchors into the arcus tendinius fascia pelvis.

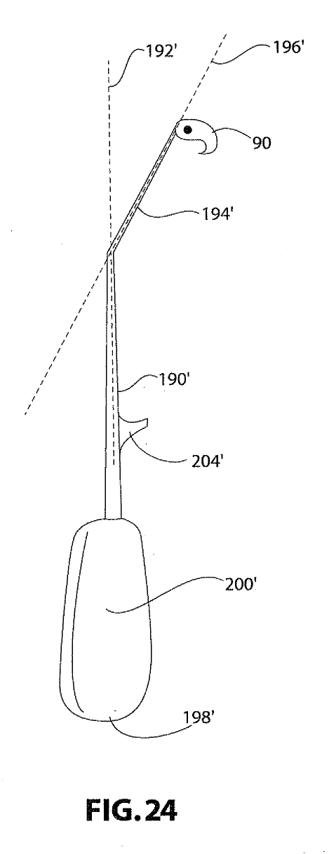

Another aspect of this embodiment, which is shown in FIG. 24, is a tool comprising a shaft having a substantially straight portion 190′. A handle 200′ is connected to the substantially straight portion 190′ at a proximal end 198′ of the shaft. At the distal end of the substantially straight portion 190′, the shaft has a curved portion 194′, which curves away from a longitudinal axis 192′ of the substantially straight portion 190′. The curved portion 194′ may comprise a second substantially straight portion. The curved portion 194′ has a distal tip 202′. A straight imaginary line 196′ drawn from the curved portion's distal tip 202′ and the distal end of the substantially straight portion 190′ has an angle between the imaginary line and the longitudinal axis of about 30 degrees to about 60 degrees, or about 45 degrees. The curved section only curves in a two dimensional plane.

In another aspect of this embodiment, the shaft has a substantially straight portion and a distal tip.

The tip is the portion of the tool adapted for receiving the base or proximal ends of anchors, or for receiving a blunt tip dissector. In one embodiment, a blunt tip dissector is connected to the tip. The blunt tip dissector is configured for attachment to the bottom of an anchor.

In certain embodiments, the tool comprises a release. The release comprises a trigger 204 that is operatively connected to the tip 202. Upon operation of the trigger 204, the tip 202 releases a component. The component is configured for removable attachment to the tip 202. The component may be a blunt tip dissector, an anchor, or an anchor comprising a blunt tip dissector. In another embodiment, the release is operatively connected to a blunt tip dissector. Upon operation of the trigger 204, the component is released from the blunt tip dissector. In this embodiment, the component may be an anchor.

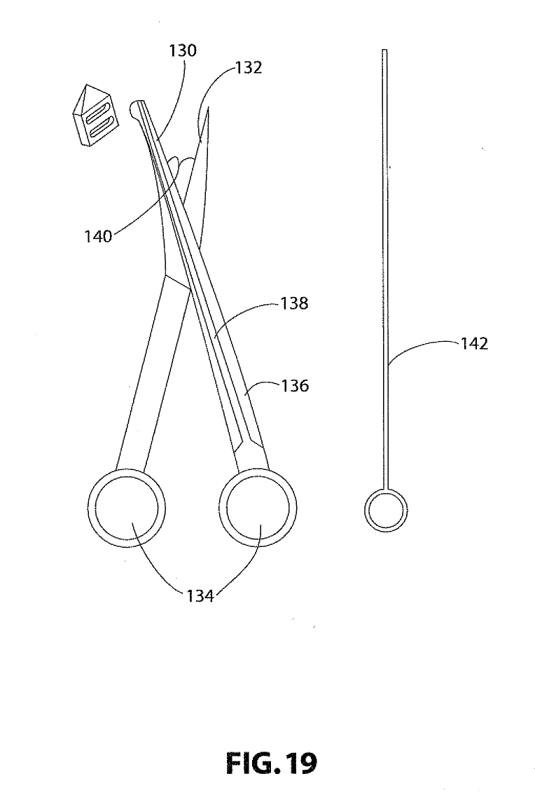

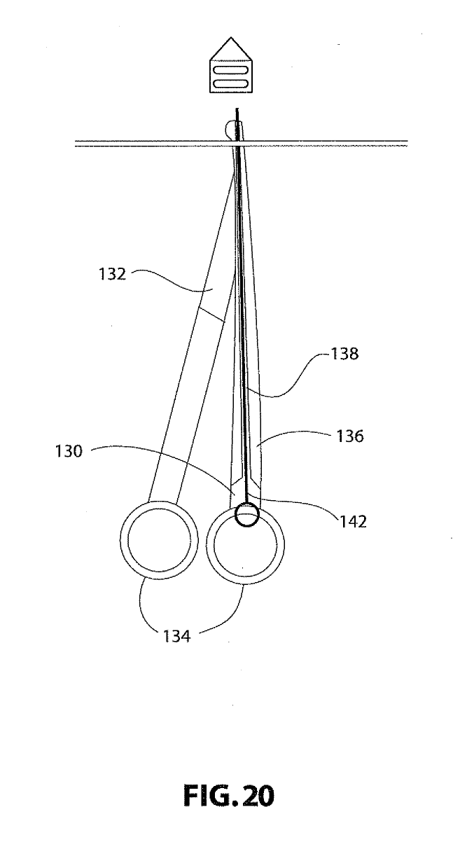

Another embodiment of this invention is scissors. The scissors comprise two cutting blades 130 and 132 hingedly connected to each other. The cutting blades have a distal end and a proximal end. Extending from the proximal end of each cutting blade 130 and 132 is a finger loop 134. The cutting blades have a cutting surface 140. Attached to at least a portion of one cutting blade at some surface other than the cutting surface is a guide 136. The guide 136 defines a channel optionally having an opening 138. The opening 138 is configured to allow the mesh 10 to be released once the anchor is implanted. In embodiments that do not comprise an opening 138, or where the opening 138 does not span the entire length of the guide 136, the anchor can be loaded into the guide at the distal end. The guide 136 allows guided passage of the anchor towards the membrane 20 (for example, the obturator membrane or the sacrospinous ligament) to place at least the head of the anchor behind or within the membrane 20. The anchor is implanted so that it remains behind or within the membrane 20. For example, upon release, the anchor may optionally change its geometric shape to remain behind or within the anchoring tissue by virtue of its resilient flexible nature. In another example, the anchor may be rotated just prior to being released. The rotation of the anchor can be accomplished by the advancing element that engages the base or bottom of the anchor. Then, the scissors inserter would be removed and adjustment would occur. Optionally, the anchor is pushed through the membrane 20 by a pushing instrument 142. The pushing instrument 142 is configured to be received by the guide 136. Once the anchor is implanted, the mesh or arm may be disengaged from the scissors by the opening 138 in the guide 136, or from the distal end of the guide 136. Examples of scissors according to this invention are provided in FIGS. 19 and 20.

Alternatively, the anchors may be implanted by an inserter. Generally, the inserter comprises a channel adapted for receiving an anchor. Optionally, the channel comprises an opening adapted to allow the mesh to be released once the anchor is implanted. The opening is further adapted to secure the anchor from releasing from the inserter.

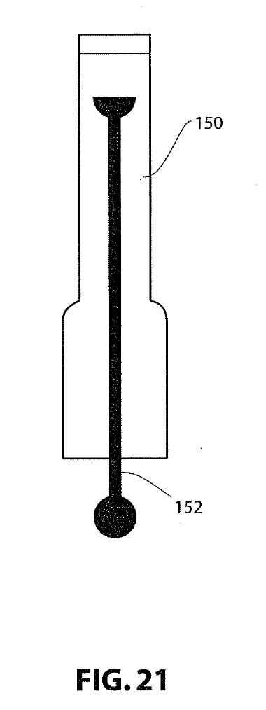

One embodiment of the inserter is depicted in FIG. 21. In this embodiment, the inserter comprises a shaft 150 defining a passage. The passage is adapted to accept and guide an anchor. The inserter further comprises a rod 152, which is adapted to push the anchor through the passage out of an opening at the distal end of the inserter. Optionally, the shaft 150 has an opening span the entire length of the shaft, or a portion of the shaft that allows the filamentary element arm or mesh to disengage the inserter. The channel passage can accept a flexible anchor in a folded state, and retain that anchor in the folded stated under the anchor is pushed through the membrane.

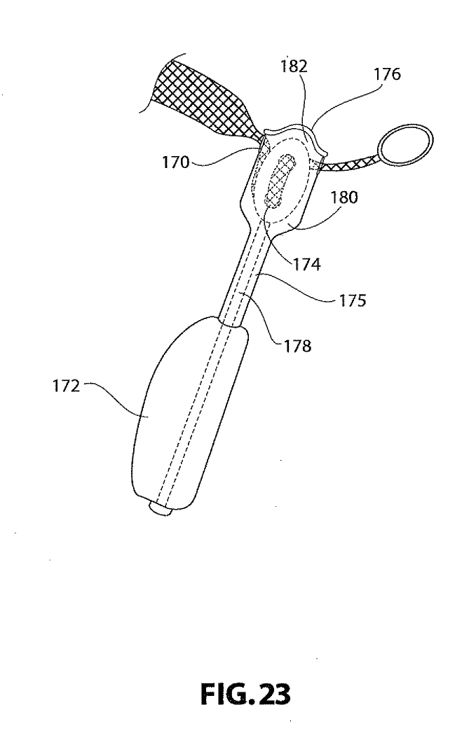

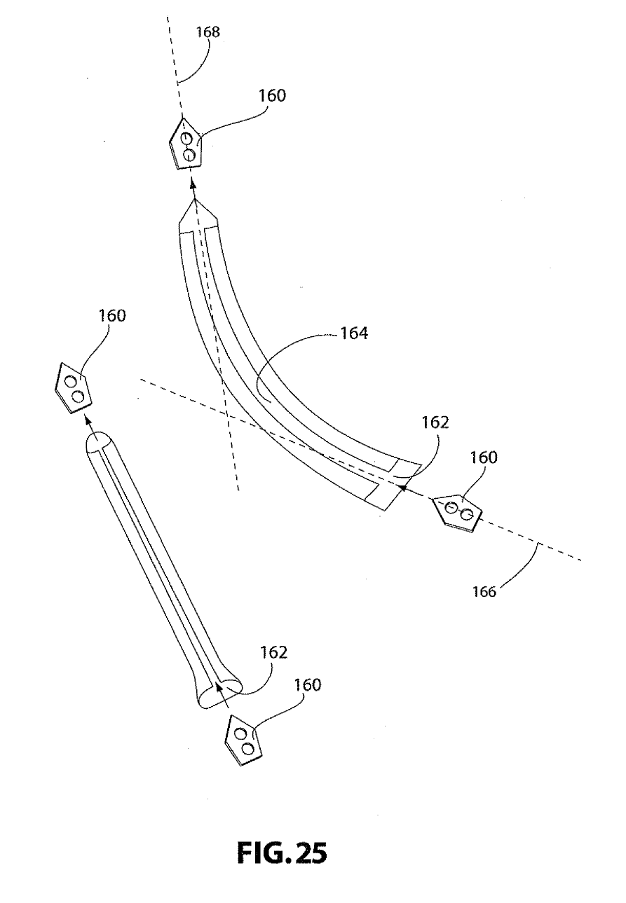

Another embodiment of the inserter is depicted in FIG. 23. In this embodiment, the inserter comprises a cradle 170 positioned at a distal end of the inserter. A handle 172 is positioned at a proximal end of the inserter. The cradle 170 defines a space 180 for an anchor 174, and comprises a distal cutting end 176 and an opening 182 positioned at or near distal cutting end 176 to accommodate the anchor 174. The inserter further comprises a pushing instrument 178 positioned proximally to the cradle 170 in communication with the space defined by the cradle 170. The pushing instrument 178 may optionally be operatively connected to a spring for biasing the pushing instrument 178 to a retracted position. When in the extended position, the pushing instrument 178 will eject the anchor from the cradle 170. When in the retracted position, the anchor can be loaded into the cradle 170. When in the retracted position, the anchor 174 can be loaded in the cradle 170. In this embodiment, the inserter has a shaft 175 that can be either can be substantially straight or curved. The curved shaft has an angle between an imaginary line extending the handle 172 and an imaginary line extending from the cradle 170 of about 20 to about 75 degrees, of about 30 to about 60 degrees, of about 40 to about 50 degrees, or of about 45 degrees, similar to the angle depicted in FIG. 25.

Another embodiment of the inserter is depicted in FIG. 25. In this embodiment, the inserter comprises a channel 162 adapted to receive an anchor 160. The channel 162 optionally comprises an opening 164 adapted to allow a mesh 10 to be removed from the inserter after the anchor 160 has been implant, and secures the anchor 160 within the channel 162. The opening 164 can traverse the entire length of the channel, or can traverse a portion of the channel. In embodiments that do not comprise an opening 164, or where the opening 164 does not span the entire length of the channel 162, the anchor can be loaded into the guide from the distal end. In this embodiment, the inserter can be substantially straight, or curved. The curved inserter has an angle between an imaginary line extending from the proximal end 166 and an imaginary line extending from the distal end 168 of about 30 to about 75 degrees, of about 30 to about 60 degrees, of about 40 to about 55 degrees, or of about 45 degrees.

Another embodiment of the invention is a kit comprising an anchor and a mesh 10 as described above. Optionally, the kit may further comprise a tool as described above. Optionally, the anchor may be positioned on a filamentary element 24 of the mesh 10, wherein the filamentary element 24 passes through the aperture or apertures of the anchor. In another embodiment, the mesh 10 comprises a clamp.

Although the present invention has been described in considerable detail with reference to preferred embodiments thereof, other embodiments are possible for those skilled in the art and various modifications and variations can be made to the present invention without departing from the scope or spirit of the invention.

1. A surgical soft-tissue anchor comprising a distal end, a proximal end opposite the distal end, a non-tissue side, a tissue side opposite the non-tissue side, a first aperture positioned between the distal end and the proximal end and extending through the anchor from the non-tissue side to the tissue side and a second aperture positioned between the first aperture and the distal end and extending through the anchor from the non-tissue side to the tissue side, wherein the distal end comprises a cutting surface adapted to cut a soft tissue, wherein the first aperture is configured to allow free movement of a filamentary element through the first aperture, and wherein the second aperture is configured to allow free movement of a filamentary element through the second aperture.

2. (canceled)

3. (canceled)

4. (canceled)

5. (canceled)

6. The surgical soft-tissue anchor of claim 1, wherein the first aperture or the second aperture is a cone-shaped aperture.

7. The surgical soft-tissue anchor of claim 1, wherein the first aperture or the second aperture comprises a rough surface.

8. The surgical soft-tissue anchor of claim 1, wherein the first aperture has an opening on the tissue side that is larger than an opening of the first aperture on the non-tissue side, and wherein the second aperture has an opening on the non-tissue side that is larger than an opening of the second aperture on the tissue side.

9. (canceled)

10. (canceled)

11. (canceled)

12. (canceled)

13. The surgical soft-tissue anchor of claim 1 further comprising a filamentary element passing through the first aperture, passing over a portion of the anchor between the first aperture and the second aperture and passing through the second aperture, wherein the filamentary element comprises a mesh at a first terminal end of the filamentary element.

14. The surgical soft-tissue anchor of claim 13, wherein the filamentary element further comprises a finger grip positioned at a second terminal end of the filamentary element.

15. The surgical soft-tissue anchor of claim 13, wherein the mesh comprises a biodegradable sheath that encases at least a portion of the mesh.

16. The surgical soft-tissue anchor of claim 15, wherein the mesh further comprises a rough edge, and wherein the biodegradable sheath comprises a perforation configured to expose the rough edge once the perforation is removed.

17. (canceled)

18. The surgical soft-tissue anchor of claim 13, wherein the filamentary element further comprises a finger grip positioned at a second terminal end of the filamentary element.

19. The surgical soft-tissue anchor of claim 1 further comprising a resilient flex line, wherein the anchor is constructed of a flexibly resilient material and can flex about the resilient flex line when acted upon.

20. An implantable supportive mesh comprising a support section, a filamentary element extending the mesh, the filamentary element extends through a first aperture of a soft-tissue anchor, the first aperture being positioned between a distal end and a proximal end of the anchor, the filamentary element extends through a second aperture, the second aperture being positioned between the first aperture and the distal end and anchor comprising a non-tissue side and a tissue side, wherein the distal end comprises a cutting surface adapted to cut through a soft tissue.

21. (canceled)

22. (canceled)

23. (canceled)

24. (canceled)

25. The implantable supportive mesh of claim 20, wherein the first aperture or second aperture is a cone-shaped aperture.

26. The implantable supportive mesh of claim 20, wherein the first aperture has an opening on the tissue side that is larger than an opening of first aperture on the non-tissue side, and wherein the second aperture has an opening on the non-tissue side that is larger than an opening of the second aperture on the tissue side.

27. (canceled)

28. (canceled)

29. The implantable supportive mesh of claim 20 further comprising a biodegradable sheath that encases at least a portion of the mesh, and a rough edge positioned on at least a portion of the mesh, and wherein the biodegradable sheath comprises a perforation configured to expose at least a portion of the rough edge once the perforation is removed.

30. (canceled)

31. The implantable supportive mesh of claim 20 further comprising an arm extending from the mesh positioned between the mesh and the filamentary element, the arm being constructed of substantially similar material as the mesh, and a biodegradable sheath that encases at least a portion of the arm.

32. The implantable supportive mesh of claim 20, wherein the filamentary element further comprises a finger grip positioned at a terminal end of the filamentary element.

33. The implantable supportive mesh of claim 26, wherein the filamentary element passes over a portion of the anchor between the first aperture and the second aperture, and passes through the second aperture.

34. (canceled)

35. (canceled)

36. (canceled)

37. (canceled)

38. (canceled)

39. (canceled)

40. (canceled)

41. (canceled)

42. A surgical kit comprising a soft-tissue anchor, an implantable supportive mesh, and a surgical tool, wherein the surgical soft-tissue anchor comprises a distal end, a proximal end opposite the distal end, a non-tissue side, a tissue side opposite the non-tissue side, a first aperture positioned between the distal end and the proximal end, the first aperture extends through the anchor from the non-tissue side to the tissue side, and a second aperture positioned between the first aperture and the distal end, the second aperture extends through the anchor from the non-tissue side to the tissue side wherein the first aperture is configured to allow free movement of a filamentary element through the first aperture; wherein the second aperture is configured to allow free movement of a filamentary element through the second aperture; and wherein the implantable supportive mesh comprises a support section, a filamentary element extending the mesh, the filamentary element is configured to be accepted by the first aperture; and a surgical tool for implanting the anchor.

43. The surgical kit according to claim 42, wherein a portion of the filamentary element is retained by the aperture.
